(19) United States
(12) Patent Application Publication
(10) Pub. No.: US 2026/0346042 A1
(43) Pub. Date: Nov. 5, 2026

(54) OPTICAL STIMULATION SYSTEMS AND METHODS FOR IMPLANTING AND USING

(71) Applicants: Alim-Louis BENABID, Meylan (FR); Stephan CHABARDES, Corenc (FR); Claude CHABROL, Poisat (FR)

(72) Inventors: Alim-Louis BENABID, Meylan (FR); Stephan CHABARDES, Corenc (FR); Claude CHABROL, Poisat (FR)

(21) Appl. No.: 19/142,729

(22) PCT Filed: Dec. 29, 2022
(86) PCT No.: PCT/IB2022/000735

§ 371 Date: Jun. 24, 2025

(51) Int. Cl. A61N 5/06

(52) U.S. Cl. CPC A61N 5/0601; A61N 5/0622; A61N 2005/0612; A61N 2005/0628; A61N 2005/063

(57) ABSTRACT

An optical stimulation system includes a light source; an optical lead coupled, or coupleable, to the light source, wherein the optical lead includes a first optical waveguide to receive light generated by the light source and emit the light from a distal portion of the optical lead; and a control unit coupled, or coupleable, to the light source and including a memory, and a processor coupled to the memory and configured for receiving, for each of a plurality of duty cycle levels, at least one parameter defining the duty cycle level, and directing the light source, using the at least one parameter of each of the duty cycle levels, to generate light in a pattern according to the plurality of duty cycle levels.

### CROSS-REFERENCE TO RELATED APPLICATIONS

This application claims the benefit under 35 U.S.C. § 119(e) of U.S. Provisional Patent Application Ser. No. 62/647,565, filed Mar. 23, 2018, which is incorporated herein by reference.

### FIELD

The present disclosure is directed to the area of implantable optical stimulation systems and methods of making and using the systems. The present disclosure is also directed to implantable optical stimulation systems utilizing therapy cycling, as well as methods of making and using the optical stimulation systems.

### BACKGROUND

Implantable optical stimulation systems can provide therapeutic benefits in a variety of diseases and disorders. For example, optical stimulation can be applied to the brain either externally or using an implanted stimulation lead to provide, for example, deep brain stimulation, to treat a variety of diseases or disorders. Optical stimulation may also be combined with electrical stimulation.

Stimulators have been developed to provide therapy for a variety of treatments. A stimulator can include a control module (for generating light or electrical signals sent to light sources in a lead), one or more leads, and one or more light sources coupled to, or disposed within, each lead. The lead is positioned near the nerves, muscles, brain tissue, or other tissue to be stimulated.

### BRIEF SUMMARY

In one aspect, an optical stimulation system includes a light source; an optical lead coupled, or coupleable, to the light source; and a control unit coupled, or coupleable, to the light source and including a memory, and a processor coupled to the memory and configured for receiving, for each of a plurality of duty cycle levels, at least one parameter defining the duty cycle level, and directing the light source, using the at least one parameter of each of the duty cycle levels, to generate light in a pattern according to the plurality of duty cycle levels.

In at least some aspects, the optical stimulation system further includes a light monitor coupled, or coupleable to, the control unit, wherein the optical lead further includes a first optical waveguide to receive light generated by the light source and emit the light from a distal portion of the optical lead and a second optical waveguide configured to receive a portion of the light emitted from the distal portion of the optical lead and direct the received portion of the light to the light monitor.

In at least some aspects, the at least one parameter of the duty cycle level includes an on duration and an off duration. In at least some aspects, the processor is further configured for displaying a user interface for duty cycle level programming. In at least some aspects, the user interface for duty cycle level programming includes controls for entry of the on duration and the off duration of each of the duty cycle levels. In at least some aspects, the user interface for duty cycle level programming includes controls for entry of the units of time for the on duration and the off duration of each of the duty cycle levels. In at least some aspects, the user interface for duty cycle level programming includes controls for turning each of the duty cycle levels on or off.

In at least some aspects, the at least one parameter of the duty cycle level includes a percentage of on time or a percentage of off time. In at least some aspects, directing the light source includes directing the light source to generate light only when all active ones of the duty cycle levels are in an on state. In at least some aspects, directing the light source includes directing the light source to not generate light when any one or more of the active ones of the duty cycle levels are in an off state.

In another aspect, a non-transitory processor readable storage media includes instructions for optical stimulation using an optical stimulation system including a light source and an optical lead, wherein execution of the instructions by one or more processor devices performs actions, including receiving, for each of a plurality of duty cycle levels, at least one parameter defining the duty cycle level, and directing the light source, using the at least one parameter of each of the duty cycle levels, to generate light in a pattern according to the plurality of duty cycle levels.

In a further aspect, a method for optical stimulation using an optical stimulation system including a light source and an optical lead, includes receiving, for each of a plurality of duty cycle levels, at least one parameter defining the duty cycle level, and directing the light source, using the at least one parameter of each of the duty cycle levels, to generate light in a pattern according to the plurality of duty cycle levels.

In at least some aspects of the non-transitory processor readable storage media or the method, the at least one parameter of the duty cycle level includes an on duration and an off duration. In at least some aspects of the non-transitory processor readable storage media or the method, the actions or steps further include displaying a user interface for duty cycle level programming. In at least some aspects of the non-transitory processor readable storage media or the method, the user interface for duty cycle level programming includes controls for entry of the on duration and the off duration of each of the duty cycle levels. In at least some aspects of the non-transitory processor readable storage media or the method, the user interface for duty cycle level programming includes controls for entry of the units of time for the on duration and the off duration of each of the duty cycle levels. In at least some aspects of the non-transitory processor readable storage media or the method, the user interface for duty cycle level programming includes controls for turning each of the duty cycle levels on or off.

In at least some aspects of the non-transitory processor readable storage media or the method, the at least one parameter of the duty cycle level includes a percentage of on time or a percentage of off time. In at least some aspects of the non-transitory processor readable storage media or the method, directing the light source includes directing the light source to generate light only when all active ones of the duty cycle levels are in an on state. In at least some aspects of the non-transitory processor readable storage media or the method, directing the light source includes directing the light source to not generate light when any one or more of the active ones of the duty cycle levels are in an off state.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic overview of one embodiment of components of an optical or optical/electrical stimulation system, including an electronic subassembly;

FIG. 2 is a schematic side view of one embodiment of an arrangement including a light source, an optional light monitor, an optical lead, and a connector lead;

FIG. 3 is a schematic cross-sectional view of one embodiment of the optical lead of FIG. 2;

FIG. 4A is a schematic side view of one embodiment of a control module configured to electrically couple to a lead or lead extension;

FIG. 4B is a schematic side view of one embodiment of a lead extension configured to electrically couple a lead to the control module of FIG. 4A;

FIG. 5 is a schematic side view of one embodiment of an electrical stimulation system that includes an electrical stimulation lead electrically coupled to a control module;

FIG. 6 is a schematic side view of one embodiment of an optical/electrical stimulation system with an optical/electrical stimulation lead coupled to a control module having a light source;

FIG. 7 is a schematic overview of one embodiment of components of a programming unit for an optical or optical/electrical stimulation system;

FIG. 8A is a schematic illustration of three different duty cycle levels for optical stimulation with a composite stimulation pattern utilizing all three duty cycle levels;

FIG. 8B is a schematic illustration of four different duty cycle levels for optical stimulation with a composite stimulation pattern utilizing all four duty cycle levels;

FIG. 9A is schematic illustration of one embodiment of a user interface for programming duty cycle levels;

FIG. 9B is schematic illustration of another embodiment of a user interface for programming duty cycle levels; and

FIG. 10 is a flowchart of one method of using an optical stimulation system.

### DETAILED DESCRIPTION

The present disclosure is directed to the area of implantable optical stimulation systems and methods of making and using the systems. The present disclosure is also directed to implantable optical stimulation systems utilizing therapy cycling, as well as methods of making and using the optical stimulation systems.

In some embodiments, the implantable optical stimulation system only provides optical stimulation. In other embodiments, the stimulation system can include both optical and electrical stimulation. In at least some of these embodiments, the optical stimulation system can be a modification of an electrical stimulation system to also, or instead, provide optical stimulation. Optical stimulation may include, but is not necessarily limited to, stimulation resulting from response to particular wavelengths or wavelength ranges of light or from thermal effects generated using light or any combination thereof.

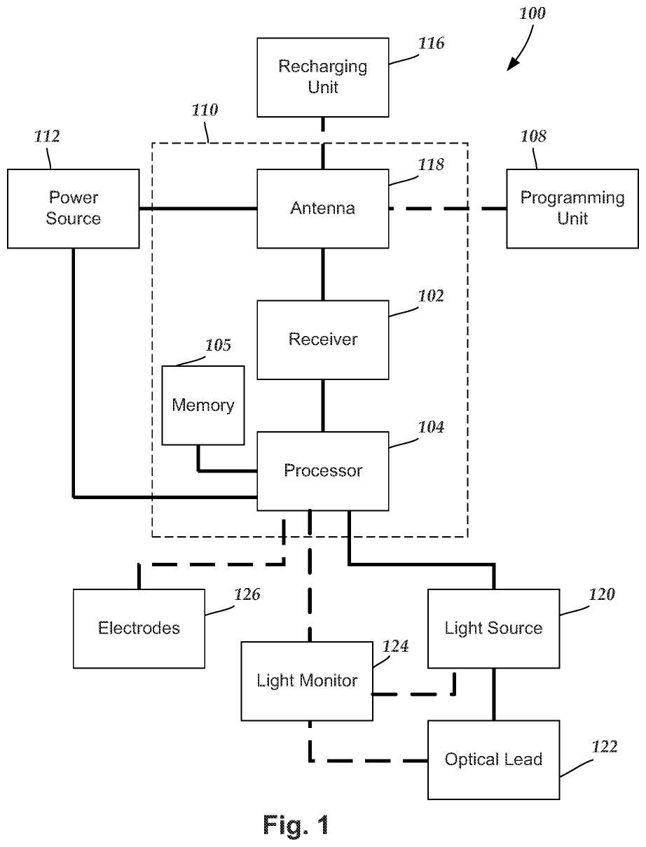

FIG. 1 is a schematic overview of one embodiment of components of an optical stimulation system 100 (or combination optical/electrical stimulation system) including an electronic subassembly 110 disposed within a control module (for example, an implantable or external pulse generator or implantable or external light generator). It will be understood that the optical stimulation system can include more, fewer, or different components and can have a variety of different configurations including those configurations disclosed in the stimulator references cited herein. In at least some embodiments, the optical stimulation system may also be capable of providing electrical stimulation through optional electrodes 126.

In at least some embodiments, selected components (for example, a power source 112, an antenna 118, a receiver 102, a processor 104, and a memory 105) of the optical stimulation system can be positioned on one or more circuit boards or similar carriers within a sealed housing of a control module. Any suitable processor 104 can be used and can be as simple as an electronic device that, for example, produces signals to direct or generate optical stimulation at a regular interval or the processor can be capable of receiving and interpreting instructions from an external programming unit 108 that, for example, allows modification of stimulation parameters or characteristics.

The processor 104 is generally included to control the timing and other characteristics of the optical stimulation system. For example, the processor 104 can, if desired, control one or more of the timing, pulse frequency, amplitude, and duration of the optical stimulation. In addition, the processor 104 can select one or more of the optional electrodes 126 to provide electrical stimulation, if desired. In some embodiments, the processor 104 selects which of the optional electrode(s) are cathodes and which electrode(s) are anodes.

Any suitable memory 105 can be used. The memory 105 illustrates a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, magnetic storage devices, or any other medium which can be used to store the desired information and which can be accessed by a processor.

The processor 104 is coupled to a light source 120. Any suitable light source can be used including, but not limited to, light emitting diodes (LEDs), organic light emitting diodes (OLEDs), laser diodes, lamps, light bulbs, or the like or any combination thereof. In at least some embodiments, the optical stimulation system may include multiple light sources. In at least some embodiments, each of the multiple light sources may emit light having a different wavelength or different wavelength range. Any suitable wavelength or wavelength range can be used including, but not limited to, visible, near infrared, and ultraviolet wavelengths or wavelength ranges. In at least some embodiments, the optical stimulation system includes a light source that emits in the orange, red, or infrared wavelength ranges (for example, in the range of 600 to 1200 nm or in the range of 600 to 700 nm or in the range of 610 to 650 nm or 620 nm or the like.) In at least some embodiments, the optical stimulation system includes a light source that emits in the green or blue wavelength ranges (for example, in the range of 450 to 550 nm or in the range of 495 to 545 nm or the like.) A wavelength or wavelength range of a light source may be selected to obtain a specific therapeutic, chemical, or biological effect.

As described below, the light source 120 may be disposed within the control module or disposed external to the control module such as, for example, in a separate unit or module or as part of an optical lead. The processor 104 provides electrical signals to operate the light source 120 including, for example, directing or driving the generation of light by the light source, pulsing the light source, or the like. For example, the processor 104 can direct current from the power source 112 to operate the light source 120. In at least some embodiments, the light source 120 is coupled to one or more optical waveguides (such as an optical fiber or other optical transmission media) disposed in an optical lead 122. In at least some embodiments, the optical lead 122 is arranged so that one or more of the optical waveguides emits light from the distal portion of the optical lead (for example, the distal end or at one or more positions along the distal portion of the lead or any combination thereof).

Optionally, the processor 104 is also coupled to a light monitor 124 that is used to monitor or measure light from the light source 122. For example, the light monitor 124 can produce electrical or other signals in response to the light received by the light monitor. Any suitable light monitor 124 can be used including, but not limited to, photodiodes, phototransistors, photomultipliers, charge coupled devices (CCDs), light dependent resistors (LRDs), photo-emissive cells, photo-conductive ells, photo-voltaic cells, photo-junction devices, or the like or any combination thereof. The light monitor 124 may be used to measure or monitor the light emitted by the light source 120 or from the optical waveguide(s) (or other optical transmission media) of the optical lead 122. In at least some embodiments, the light monitor 124 may be coupled to one or more optical waveguides (or other optical transmission media) of the optical lead 122 to transmit the light along an optical lead for measurement or monitoring.

Any power source 112 can be used including, for example, a battery such as a primary battery or a rechargeable battery. Examples of other power sources include super capacitors, nuclear or atomic batteries, fuel cells, mechanical resonators, infrared collectors, flexural powered energy sources, thermally-powered energy sources, bioenergy power sources, bioelectric cells, osmotic pressure pumps, and the like. As another alternative, power can be supplied by an external power source through inductive coupling via an antenna 118 or a secondary antenna. The external power source can be in a device that is mounted on the skin of the user or in a unit that is provided near the user on a permanent or periodic basis. In at least some embodiments, if the power source 112 is a rechargeable battery, the battery may be recharged using the antenna 118 and a recharging unit 116. In some embodiments, power can be provided to the battery for recharging by inductively coupling the battery to the external recharging unit 116.

In at least some embodiments, the processor 104 is coupled to a receiver 102 which, in turn, is coupled to an antenna 118. This allows the processor 104 to receive instructions from an external source, such as programming unit 108, to, for example, direct the stimulation parameters and characteristics. The signals sent to the processor 104 via the antenna 118 and the receiver 102 can be used to modify or otherwise direct the operation of the optical stimulation system. For example, the signals may be used to modify the stimulation characteristics of the optical stimulation system such as modifying one or more of stimulation duration and stimulation amplitude. The signals may also direct the optical stimulation system 100 to cease operation, to start operation, to start charging the battery, or to stop charging the battery. In other embodiments, the stimulation system does not include the antenna 118 or receiver 102 and the processor 104 operates as initially programmed.

In at least some embodiments, the antenna 118 is capable of receiving signals (e.g., RF signals) from an external programming unit 108 (such as a clinician programmer or patient remote control or any other device) which can be programmed by a user, a clinician, or other individual. The programming unit 108 can be any unit that can provide information or instructions to the optical stimulation system 100. In at least some embodiments, the programming unit 108 can provide signals or information to the processor 104 via a wireless or wired connection. One example of a suitable programming unit is a clinician programmer or other computer operated by a clinician or other user to select, set, or program operational parameters for the stimulation. Another example of the programming unit 108 is a remote control such as, for example, a device that is worn on the skin of the user or can be carried by the user and can have a form similar to a pager, cellular phone, or remote control, if desired. In at least some embodiments, a remote control used by a patient may have fewer options or capabilities for altering stimulation parameters than a clinician programmer.

Optionally, the optical stimulation system 100 may include a transmitter (not shown) coupled to the processor 104 and the antenna 118 for transmitting signals back to the programming unit 108 or another unit capable of receiving the signals. For example, the optical stimulation system 100 may transmit signals indicating whether the optical stimulation system 100 is operating properly or not or indicating when the battery needs to be charged or the level of charge remaining in the battery. The processor 104 may also be capable of transmitting information about the stimulation characteristics so that a user or clinician can determine or verify the characteristics.

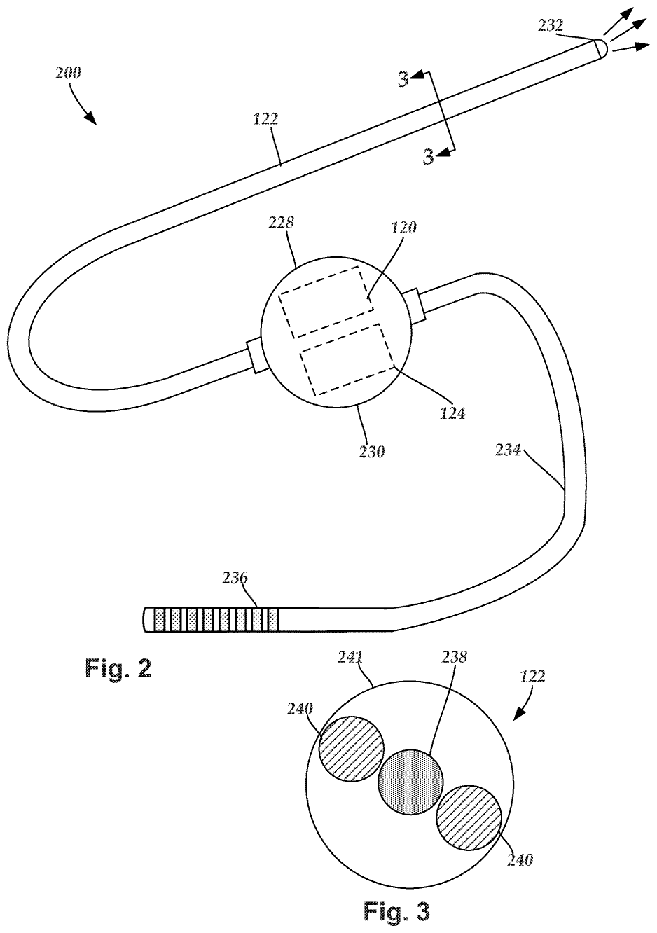

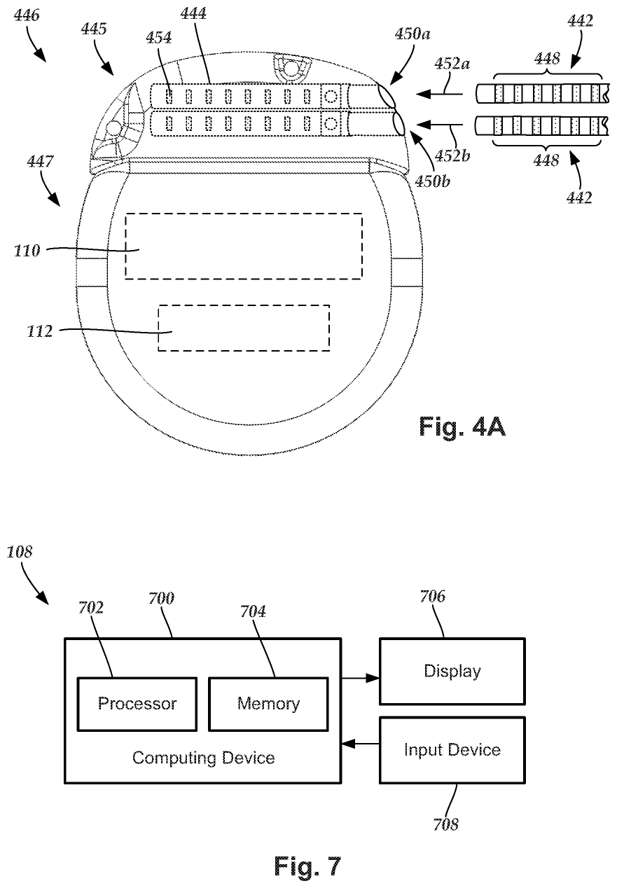

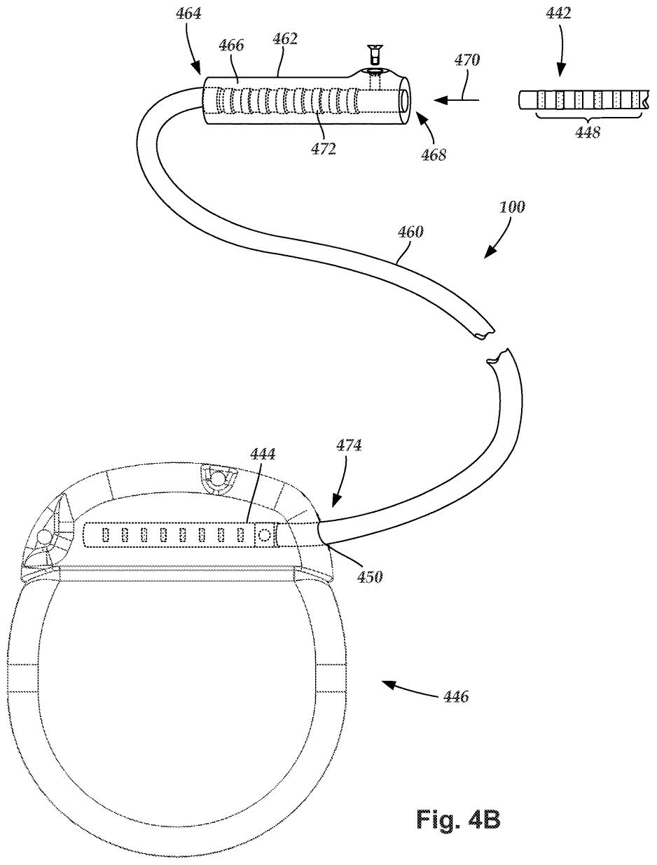

FIG. 2 illustrates one embodiment of an arrangement 200 for an optical stimulation system that can be used with a control module (see, FIG. 4). In at least some embodiments, the control module may be originally designed for use with an electrical stimulation system and adapted for use as an optical stimulation system via the arrangement 200.

The arrangement 200 includes a base unit 228 a light source 120 disposed in a housing 230, an optical lead 122 with one or more emission regions 232a, 232b of a distal portion from which light is emitted, and a connector lead 234 with one or more terminals 236 for coupling to a control module or lead extension, as described below. The optical lead 122 and connector lead 234, independently, may be permanently, or removably, coupled to the base unit 228. If removably coupleable to the base unit 228, the optical lead 122, connector lead 234, or both will have corresponding arrangements (for example, terminals and contacts) for transmission of light (for the optical lead) or electrical signals (for the connector lead) to the base unit 228. The one or more emission regions 232a, 232b may include a tip emission region 232a that emits distally away from the lead or may include a side emission regions 232b that emit at the sides of the lead or any combination thereof.

In addition to the light source 120, the base unit 228 can optionally include a light monitor 124. The base unit 228 may also include components such as electrical components associated with the light source 120 or light monitor 124, a heat sink, optical components (for example, a lens, polarizer, filter, or the like), a light shield to reduce or prevent light emission out of the housing of the base unit or to reduce or prevent extraneous light from penetrating to the light monitor 124 or the like. The housing 230 of the base unit 228 can be made of any suitable material including, but not limited to, plastic, metal, ceramic, or the like, or any combination thereof. If the base unit 228 is to be implanted, the housing 230 is preferably made of a biocompatible material such as, for example, silicone, polyurethane, titanium or titanium alloy, or any combination thereof.

In at least some embodiments, the optical lead 122, as illustrated in cross-section in Figured 3, includes a lead body 241 and one or more optical waveguides 238 (or other optical transmission media) for transmission of light from the light source 120 with emission along the one or more emission regions 232a, 232b disposed on the distal portion of the optical lead. In the illustrated embodiment, the light is emitted at the distal tip of the lead. In other embodiments, the light may be emitted at one or more points along the length of at least the distal portion of the lead. In some embodiments with multiple light sources, there may be separate optical waveguides for each light source or light from multiple light sources may be transmitted along the same optical waveguide(s). The optical lead 122 may also include one or more optical components, such as a lens, diffuser, polarizer, filter, or the like, at the distal portion of the lead (for example, at the terminal end of the optical waveguide 238) to modify the light transmitted through the optical waveguide.

In at least some embodiments that include a light monitor 124, the optical lead 122 may include one or more optical waveguides 240 (or other optical transmission media) that receive light emitted from the light source 120 and transmitted by the optical waveguide 238 in order to measure or monitor the light emitted at the one or more emission regions 232a, 232b of the optical lead. The optical waveguide(s) 240 transmit light from the one or more emission regions 232a, 232b of the optical lead to the light monitor 124 in the base unit 228. The optical lead 122 may also include one or more optical components, such as a lens, diffuser, polarizer, filter, or the like, at the distal portion of the lead (for example, at the terminal end of the optical waveguide 240) to modify the light received by the optical waveguide(s) 240.

The connector lead 234 includes conductors (e.g., wires—not shown) disposed in a lead body extending along the connector lead 234 to the terminals 236 on the proximal end of the connector lead. As an alternative, the connector lead 234 may be permanently attached to a control module or other device where the conductors then attach to contact points within the control module or other device. The conductors carry electrical signals to the base unit 228 and the light source 120 and, optionally, other electrical components in the base unit for operation of the light source 120. The conductors may also carry electrical signals from the optional light monitor 124 in the base unit 228 to the control module or other device. These electrical signals may be generated by the light monitor 124 in response to light received by the light monitor.

FIG. 4A is a schematic side view of one embodiment of proximal ends 442 of one or more leads (for example, connector lead 234 of FIG. 2) or lead extensions 460 (see, FIG. 4B) coupling to a control module 446 (or other device) through one or more control module connectors 444. The one or more proximal ends 442 include terminals 448 (for example, terminals 236 of connector lead 234).

The control module connector 444 defines at least one port 450a, 450b into which a proximal end 442 can be inserted, as shown by directional arrows 452a and 452b. The control module 446 (or other device) can define any suitable number of ports including, for example, one, two, three, four, five, six, seven, eight, or more ports.

The control module connector 444 also includes a plurality of connector contacts, such as connector contact 454, disposed within each port 450a and 450b. When the proximal end 442 is inserted into the ports 450a and 450b, the connector contacts 454 can be aligned with a plurality of terminals 448 disposed along the proximal end(s) 442. Examples of connectors in control modules are found in, for example, U.S. Pat. Nos. 7,244,150 and 8,224,450, which are incorporated by reference.

The control module 446 typically includes a connector housing 445 and a sealed electronics housing 447. An electronic subassembly 110 (see, FIG. 1) and an optional power source 112 (see, FIG. 1) are disposed in the electronics housing 447.

FIG. 4B is a schematic side view of a portion of another embodiment of an optical stimulation system 100. The optical stimulation system 100 includes a lead extension 460 that is configured to couple one or more proximal ends 442 of a lead to the control module 446. In FIG. 4B, the lead extension 460 is shown coupled to a single port 450 defined in the control module connector 444. Additionally, the lead extension 460 is shown configured to couple to a single proximal end 442 of a lead (for example, the connector lead 234 of FIG. 2).

A lead extension connector 462 is disposed on the lead extension 460. In FIG. 4B, the lead extension connector 462 is shown disposed at a distal end 464 of the lead extension 460. The lead extension connector 462 includes a connector housing 466. The connector housing 466 defines at least one port 468 into which terminals 448 of the proximal end 442 of the lead can be inserted, as shown by directional arrow 470. The connector housing 466 also includes a plurality of connector contacts, such as connector contact 472. When the proximal end 442 is inserted into the port 468, the connector contacts 472 disposed in the connector housing 466 can be aligned with the terminals 448 for electrical coupling.

In at least some embodiments, the proximal end 474 of the lead extension 460 is similarly configured as a proximal end 442 of a lead. The lead extension 460 may include a plurality of electrically conductive wires (not shown) that electrically couple the connector contacts 472 to a proximal end 474 of the lead extension 460 that is opposite to the distal end 464. In at least some embodiments, the conductive wires disposed in the lead extension 460 can be electrically coupled to a plurality of terminals (not shown) disposed along the proximal end 474 of the lead extension 460. In at least some embodiments, the proximal end 474 of the lead extension 460 is configured for insertion into a connector disposed in another lead extension (or another intermediate device). In other embodiments (and as shown in FIG. 4B), the proximal end 474 of the lead extension 460 is configured for insertion into the control module connector 144.

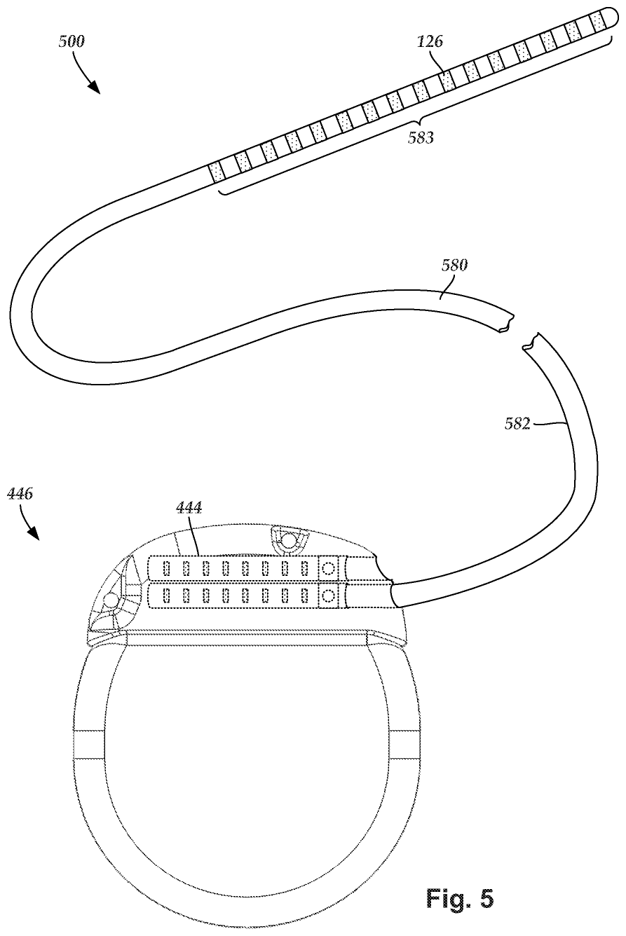

In some embodiments, the optical stimulation system may also be an electrical stimulation system. FIG. 5 illustrates schematically one embodiment of an electrical stimulation system 500. The electrical stimulation system includes a control module 446 (e.g., a stimulator or pulse generator) and an electrical stimulation lead 580 coupleable to the control module 446. The same control module 446 can be utilized with the arrangement 200 (FIG. 2) for optical stimulation and an electrical stimulation lead 580. With respect to the optical/electrical stimulation system of FIG. 1, the control module 446 can include the electronic subassembly 110 (FIG. 1) and power source 112 (FIG. 1) and the electrical stimulation lead 580 can include the electrodes 126. The optical arrangement 200 of FIG. 2 can be inserted into another port of the control module 446.

The lead 580 includes one or more lead bodies 582, an array of electrodes 583, such as electrode 126, and an array of terminals (e.g., 448 in FIG. 4A-4B) disposed along the one or more lead bodies 582. In at least some embodiments, the lead is isodiametric along a longitudinal length of the lead body 582. Electrically conductive wires, cables, or the like (not shown) extend from the terminals to the electrodes 126. Typically, one or more electrodes 126 are electrically coupled to each terminal. In at least some embodiments, each terminal is only connected to one electrode 126.

The lead 580 can be coupled to the control module 446 in any suitable manner. In at least some embodiments, the lead 580 couples directly to the control module 446. In at least some other embodiments, the lead 580 couples to the control module 446 via one or more intermediate devices. For example, in at least some embodiments one or more lead extensions 460 (see e.g., FIG. 4B) can be disposed between the lead 580 and the control module 446 to extend the distance between the lead 580 and the control module 446. Other intermediate devices may be used in addition to, or in lieu of, one or more lead extensions including, for example, a splitter, an adaptor, or the like or combinations thereof. It will be understood that, in the case where the electrical stimulation system 500 includes multiple elongated devices disposed between the lead 580 and the control module 446, the intermediate devices may be configured into any suitable arrangement.

The electrical stimulation system or components of the electrical stimulation system, including one or more of the lead bodies 582 and the control module 446, are typically implanted into the body of a patient. The electrical stimulation system can be used for a variety of applications including, but not limited to, brain stimulation, neural stimulation, spinal cord stimulation, muscle stimulation, and the like.

The electrodes 126 can be formed using any conductive, biocompatible material. Examples of suitable materials include metals, alloys, conductive polymers, conductive carbon, and the like, as well as combinations thereof. In at least some embodiments, one or more of the electrodes 126 are formed from one or more of: platinum, platinum iridium, palladium, palladium rhodium, or titanium. The number of electrodes 126 in each array 583 may vary. For example, there can be two, four, six, eight, ten, twelve, fourteen, sixteen, or more electrodes 126. As will be recognized, other numbers of electrodes 126 may also be used.

Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 6,175,710; 6,224,450; 6,271,094; 6,295,944; 6,364,278; and 6,391,985; U.S. Patent Applications Publication Nos. 2007/0150036;

2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties.

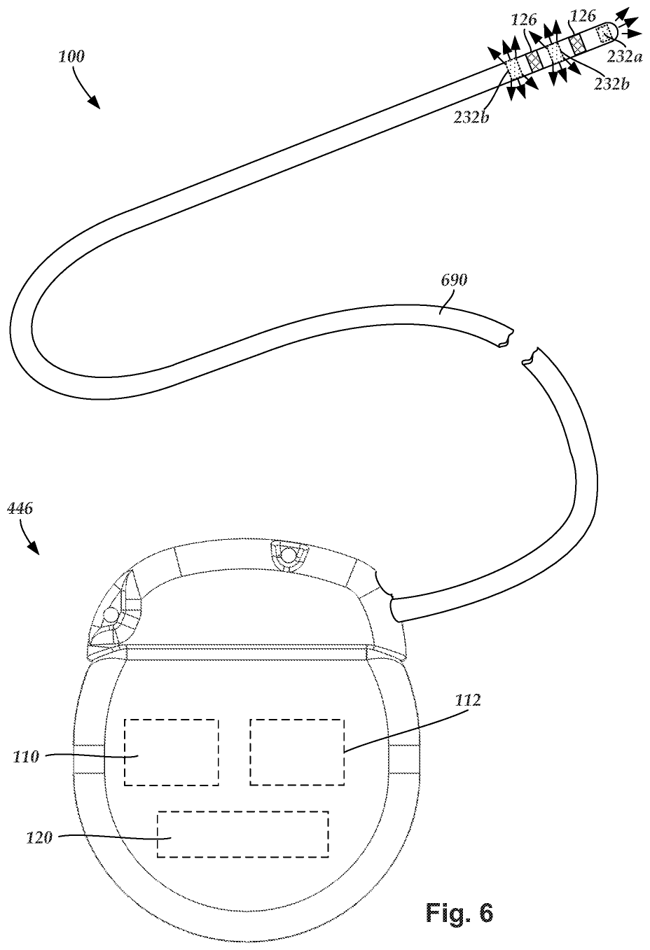

FIG. 6 illustrates other optional embodiments. For example, FIG. 6 illustrates one embodiment of an optical/electrical stimulation system 100 with a lead 690 with both electrodes 126 and an optical waveguide that emits light from the from one more emission regions 232a, 232b of the lead. In some embodiments, the lead 690 can be coupled to the base unit 228 and connector lead 234 of FIG. 2 with conductors (and optionally connector contacts if the lead 690 or connector lead 234 are removable from the base unit 228) electrically coupling the terminals 236 of the connector lead to the electrodes 126 of the lead 690.

FIG. 6 also illustrates one embodiment of a control module 446 that also includes a light source 120 within the control module. Such an arrangement can replace the base unit 228 and connector lead 234 of FIG. 2 and may include a lead extension 460.

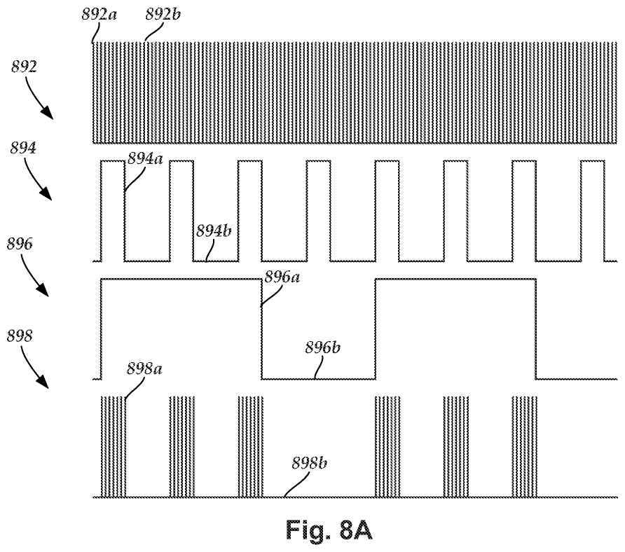

FIG. 7 illustrates one embodiment of a programming unit 108. The programming unit 108 can include a computing device 700 or any other similar device that includes a processor 702 and a memory 704, a display 706, and an input device 708.

The computing device 700 can be a computer, tablet, mobile device, or any other suitable device for processing information or programming an optical stimulation system. The computing device 700 can be local to the user or can include components that are non-local to the computer including one or both of the processor 702 or memory 704 (or portions thereof). For example, in at least some embodiments, the user may operate a terminal that is connected to a non-local computing device. In other embodiments, the memory can be non-local to the user.

The computing device 700 can utilize any suitable processor 702 including at least one hardware processors that may be local to the user or non-local to the user or other components of the computing device. The processor 702 is configured to execute instructions provided to the processor 702, as described below.

Any suitable memory 704 can be used for the computing device 702. The memory 704 illustrates a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has at least one of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The display 706 can be any suitable display device, such as a monitor, screen, display, or the like, and can include a printer. In at least some embodiments, the display 706 may form a single unit with the computing device 700. The input device 708 can be, for example, a keyboard, mouse, touch screen, track ball, joystick, voice recognition system, or any combination thereof, or the like.

The methods and systems described herein may be embodied in many different forms and should not be construed as limited to the embodiments set forth herein. Accordingly, the methods and systems described herein may take the form of an entirely hardware embodiment, an entirely software embodiment or an embodiment combining software and hardware aspects. Systems referenced herein typically include memory and typically include methods for communication with other devices including mobile devices. Methods of communication can include both wired and wireless (for example, RF, optical, or infrared) communications methods and such methods provide another type of computer readable media; namely communication media. Wired communication can include communication over a twisted pair, coaxial cable, fiber optics, wave guides, or the like, or any combination thereof. Wireless communication can include RF, infrared, acoustic, near field communication, Bluetooth™, or the like, or any combination thereof.

This optical power output by the light source 120 is often different from the optical power output at the distal end of the optical lead. For example, the components in the light source 120 and optical lead 122, as well as the manufacturing process, produce light losses from the optical power output at the distal end of the optical lead. Moreover, the individual light sources and optional light monitors, as well as other optical components such as the optical waveguides, generally have variations of performance from part to part. In addition, the power source 112 can have an associated tolerance and can vary between individual units.

As described above, an optical stimulation system provides energy to a light source to generate light for therapy. In at least some embodiments, efficacy is maintained when therapy is cycled on and off, and power is saved by cycling as compared to leaving therapy on continuously. In at least some cases, only minutes or hours of therapy are needed per day to obtain the desired therapeutic effect. For a system with a rechargeable power source, cycling allows the user to charge less often while maintaining therapy. For a system with a primary cell power source, cycling extends the life of the stimulator while maintaining therapy. In at least some embodiments, cycling at a small timescale is part of therapy and cycling at a larger time scale saves energy.

In at least some embodiments, an optical or optical/electrical stimulation system provides the user an interface or other mechanism for specifying an on duration and an off duration at multiple time scales. Such an arrangement can result in a more complicated pulsing stimulation scheme. Reduction of the optical (or optical and electrical) stimulation delivered to patient tissue over time can be useful to prolong battery life and, in the case of rechargeable power sources, reduce the frequency or duration (or both) of recharging sessions. Such reduced energy, however, preferably continues to produce the desired therapeutic effect. Such a reduction can be accomplished, with few or no side effects, by managing the duty cycle of the stimulation. The duty cycle is provided to suppress some or all of the stimulation that would have been provided if the duty cycle were not imposed. Implementation of the duty cycle may be handled by firmware, hardware, or software, or any combination thereof.

In at least some embodiments, the duty cycling can be performed at multiple levels. The duty cycle can be implemented in a variety of ways. For example, an “on” duration and an “off” duration can be specified. Alternatively, a percentage of “on time” or of “off time” can be specified.

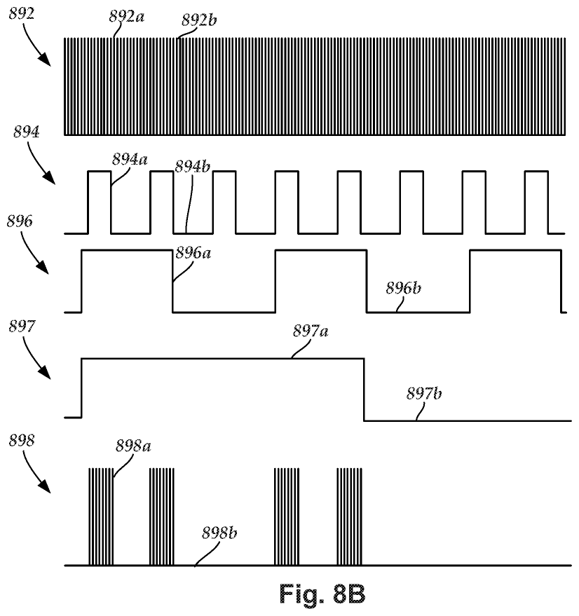

FIG. 8A illustrates three levels 892, 894, and 896 of duty cycles with on periods 892a, 894a, 896a and off periods 892b, 894b, 896b. If all three levels are implemented simultaneously, the resulting stimulation pattern 898 is obtained with on periods 898a which correspond to periods in time when the on periods 892a, 894a, 896a coincide and off periods 898b when at least one of the levels 892, 894, 896 is in an off period 892b, 894 b, 896 b. As an example, level 1 892 can have an on time of 13 milliseconds and an off time of 26 milliseconds and may represent to the periodic optical stimulation pattern. Level 2 can have an on time of 1 second and an off time of 3 seconds. Level 3 can have an on time of 1 hour and an off time of 3 hours. It will be recognized that for ease of illustration, the patterns of FIG. 8A are not to scale with the example times. An alternative understanding of this arrangement is that level 892 is the optical stimulation and levels 894 and 896 correspond to two duty cycle levels.

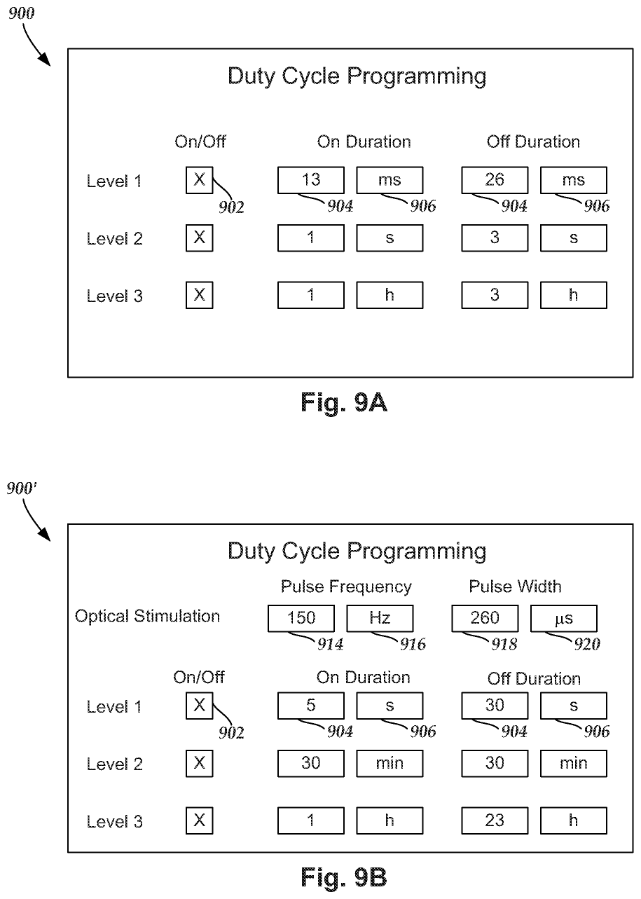

FIG. 8B illustrates four levels 892, 894, 896, and 897 of duty cycles with on periods 892a, 894a, 896a, 897a and off periods 892b, 894b, 896b, 897b. If all four duty cycles are implemented simultaneously, the resulting stimulation pattern 898 is obtained with on periods 898a which correspond to periods in time when the on periods 892a, 894a, 896a, 897a coincide and off periods 898b when at least one of the levels 892, 894, 896, 897 is in an off period 892b, 894b, 896b, 897b. As an example, level 1 892 can correspond to an optical stimulation pattern with a pulse width of, for example, 260 μs and a pulse frequency of, for example, 150 Hz. Level 2 can have an on time of 5 seconds and an off time of 60 seconds. Level 3 can have an on time of 30 minutes and an off time of 30 minutes. Level 4 can have an on-time of 1 hour and an off time of 23 hours. It will be recognized that for ease of illustration, the patterns of FIG. 8B are not to scale with the example times. An alternative understanding of this arrangement is that level 892 is the optical stimulation and levels 894, 896, and 897 correspond to three duty cycle levels (see, FIG. 9B).

The duty cycle for the different levels may be selected to manage different aspects of the stimulation and device. Examples of such aspects include, but are not limited to, optical source efficiency, thermal management (for example, in the case of high laser power), battery management, therapy management (for example, the amount of light received by the patient or the time during which the patient receives therapy). Each of the cycling levels may be used by the same or different users (neurologist, clinician programmer, or the like) to address these considerations. For example, one level may be selected for thermal or heat management to reduce or prevent overheating of tissue, light source, or other components of the system. A level may be selected for battery management in order to increase the time between recharges or to prolong the battery. Preferably, the duty cycle imposed for battery management maintains the therapeutic benefits at a desired level while reducing battery use. Another level may be selected for therapy management to, for example, allow recovery periods between therapy sessions or to reflect that therapy may be achieved during an on period and then maintained during a subsequent off period. In some embodiments, each level may be password protected or require authentication to change.

The duration of the on and off periods for each duty cycle level can be defined in the system (for example, in the programming unit 108 or the control module 446.) In at least some embodiments, the programming unit 108 can include a user interface presented on the display 706 (FIG. 7) which allows a user (such as a clinician, programmer, patient, or other individual) to select the durations of the on and off periods for each duty cycle level and may also allow the user to turn on/off each individual duty cycle level. Examples interfaces that can be modified to include such user-input can be found in, for example, U.S. Pat. Nos. 8,326,433; 8,831,731; 8,849,632; 9,050,470; and 9,072,905; and U.S. Patent Application Publication No. 2014/0277284, all of which are incorporated herein by reference in their entireties.

One example of a user interface 900 is illustrated in FIG. 9A for the arrangement illustrated in FIG. 8A. The user interface 900 includes multiple duty cycle levels with level controls 902 for selecting whether the level is on or off, on-time controls 904 for selecting the values of time durations, and off-time controls 906 for selecting the units (for example, microseconds (μs), milliseconds (ms), seconds(s), minutes (m), hours (h), days (d), or weeks (w) or any other suitable time period) for each of these durations. If the duty cycle is described in terms of a percentage of the time on or off, suitable controls for such a selection can be used instead or, or in addition to, those illustrated in FIG. 9A. In some embodiments, selection of the duration values can be used by text entry, up/down controls, pre-defined lists of values, a clock style control, a slider bar, or any other suitable input mechanism. In some embodiments, the user interface can include more than three (or less than three) duty cycle levels or may include controls allowing the user to add additional duty cycle levels.

In at least some embodiments, the user interface 900 may also include representations of one or more of the three levels 892, 894, and 896 in a time diagram, as shown in FIG. 8A. In at least some embodiments, the user interface 900 may also include a representation of the stimulation pattern 898 in a time diagram, as shown in FIG. 8A.

One example of a user interface 900′ is illustrated in FIG. 9B for the arrangement illustrated in FIG. 8B. The user interface 900 includes stimulation controls (or informational displays if the stimulation is defined elsewhere in the user interface) such as, for example, pulse frequency controls 914, 916 for defining a pulse frequency and pulse width controls 918, 920 for defining a pulse width. The user interface also includes multiple duty cycle levels with level controls 902 for selecting whether the duty cycle level is on or off, on-time controls 904 for selecting the values of time durations, and off-time controls 906 for selecting the units (for example, microseconds (μs), milliseconds (ms), seconds(s), minutes (m), hours (h), days (d), or weeks (w) or any other suitable time period) for each of these durations. If the duty cycle is described in terms of a percentage of the time on or off, suitable controls for such a selection can be used instead or, or in addition to, those illustrated in FIG. 9B. In some embodiments, selection of the duration values can be used by text entry, up/down controls, pre-defined lists of values, a clock style control, a slider bar, or any other suitable input mechanism. In some embodiments, the user interface can include more than three (or less than three) duty cycle levels or may include controls allowing the user to add additional duty cycle levels.

In at least some embodiments, the user interface 900 may also include representations of one or more of the four levels 892, 894, 896, and 897 in a time diagram, as shown in FIG. 8B. In at least some embodiments, the user interface 900 may also include a representation of the stimulation pattern 898 in a time diagram, as shown in FIG. 8B.

It will be understood that the arrangement of FIG. 8A can also be adapted to the user interface 900′ of FIG. 9B which would include one set of stimulation controls and two sets of duty cycle controls. It will be understood that the arrangement of FIG. 8B can also be adapted to the user interface 900 of FIG. 9A which would include 4 sets of level controls.

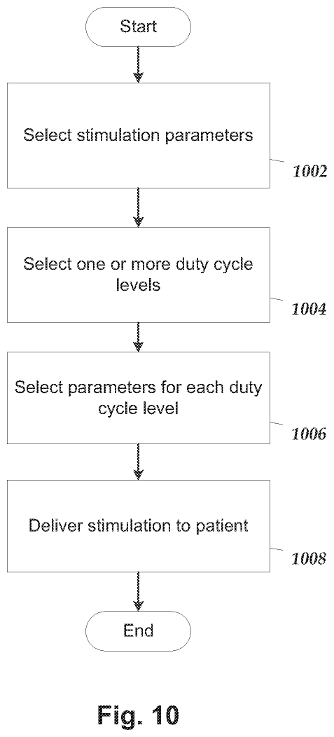

FIG. 10 illustrates one example of a method for delivering optical stimulation to patient tissue. In step 1002, stimulation parameters such as stimulation amplitude (or pulse amplitude), stimulation duration, or any combination thereof can be selected by a practitioner or a patient or any other suitable user. The permission to select any of the parameters described in this paragraph or below may be limited to any subset of the practitioner, patient, or others. For example, in some embodiments, the patient is given permission to set or alter parameters. In other embodiments, the patient is not given permission to set or alter one or more (or even all) of the parameters. It will be understood that other stimulation parameters can be selected and that more complex stimulation patterns may include selection of more than one value for each parameter.

In step 1004, one or more duty cycle levels are selected by a practitioner or a patient or any other suitable user. In step 1006, parameters, such as on duration and off duration, are selected for each duty cycle level by a practitioner or a patient or any other suitable user. In step 1008, optical stimulation is delivered to patient tissue as a series of pulses from the light source through an implanted optical lead according to the duty cycle levels and parameters selected in steps 1002, 1004, and 1006. It will also be recognized that the optical stimulation may be delivered in conjunction with electrical stimulation which may, or may not, utilize the same duty cycle levels and associated parameters.

It will be understood that each block of the flowchart illustrations, and combinations of blocks in the flowchart illustrations and methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, at least one process may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

A system can include one or more processors that can perform the methods (in whole or in part) described above. The methods, systems, and units described herein may be embodied in many different forms and should not be construed as limited to the embodiments set forth herein. Accordingly, the methods, systems, and units described herein may take the form of an entirely hardware embodiment, an entirely software embodiment or an embodiment combining software and hardware aspects. The methods described herein can be performed using any type of processor or any combination of processors where each processor performs at least part of the process. In at least some embodiments, the processor may include more than one processor.

The above specification provides a description of the manufacture and use of the invention. Since many embodiments of the invention can be made without departing from the spirit and scope of the invention, the invention also resides in the claims hereinafter appended.

1. A method for implanting and using an optical stimulation system comprising an optical lead, a base unit comprising a light source, and a control module, wherein the base unit is coupleable, or coupled, to the optical lead and the control module, the method comprising:
forming a first burr hole in a skull of a patient for implantation of the optical lead; forming a second burr hole in the skull and spaced apart from the first burr hole and configured to receive at least a portion of the base unit;
coupling the optical lead to the base unit;
coupling the base unit to the control module;
implanting the optical lead through the first burr hole into the skull of the patient; and receiving the portion of the base unit into the second burr hole.

2. The method of claim 1, wherein the second burr hole has a larger diameter than the first burr hole.

3. The method of claim 1, further comprising fastening the base unit to the skull.

4. The method of claim 1, further comprising implanting the control module in the patient.

5. The method of claim 4, wherein the implanting comprises implanting the control module in a subclavicular region of the patient.

6. The method of claim 1, further comprising tunneling at least a connector lead from the base unit to the control module.

7. The method of claim 1, further comprising, prior to implanting the optical lead, inserting a cannula through the first burr hole into the brain of the patient, wherein the implanting comprises implanting the optical lead through the cannula.

8. The method of claim 1, further comprising fastening the optical lead to the skull and the implanting.

9. The method of claim 1, further comprising after the implanting, optically stimulating the patient using the optical stimulation system.

10. The method of claim 9, further comprising after the optically stimulating, obtaining a PET scan to assess effectiveness of the optical stimulation.

11. A method for implanting an optical stimulation system comprising an optical lead, a base unit comprising a light source, and a control module, wherein the base unit is coupleable, or coupled, to the optical lead and the control module, the method comprising:
coupling the base unit to the control module prior to implanting the optical lead; coupling the optical lead to the base unit prior to implanting the optical lead;
testing the optical lead and the light source in the base unit using the control module to determine that power is delivered from the control module to the light source, light is produced by light source and delivered to the optical lead, and the light is emitted from the optical lead at at least one emission region disposed on a distal portion of the optical lead; and
after the testing, implanting the optical lead into a brain of a patient.

12. The method of claim 11, further comprising, prior to the testing, implanting the control module into the patient.

13. The method of claim 11, further comprising, prior to the testing, implanting at least a portion the base unit into the patient.

14. The method of claim 11, wherein the testing comprises determining a value of an optical power of light emitted from the at least one emission region of the optical lead for a plurality of different light source current values.

15. The method of claim 14, wherein the testing further comprises using the values of optical power determined by the testing in a comparison test based on previously measured optical power values.

16. The method of claim 15, wherein the comparison test comprises comparing a slope of the values of optical power determined by the testing with a slope of the previously measured optical power values.

17. A method for implanting an optical stimulation system comprising an optical lead, a base unit comprising a light source, and a control module, wherein the base unit is coupleable, or coupled, to the optical lead and the control module, the method comprising:
forming a first burr hole in a skull of a patient for implantation of the optical lead; inserting a cannula through the first burr hole into a brain of the patient;
delivering a contrast agent through the cannula to the brain of the patient;
visualizing at least one ventricle of the brain of the patient using the contrast agent; and after the visualizing, implanting the optical lead through the cannula into the brain of the patient using the visualization to guide placement of the optical lead.

18. The method of claim 17, further comprising, prior to implantation surgery, imaging the brain of the patient.

19. The method of claim 18, further comprising, prior to implantation surgery, using the imaging to plan a trajectory for the optical lead into the brain of the patient.

20. The method of claim 17, further comprising using the trajectory or an actual position of the optical lead in the brain of the patient to estimate an optical power of the optical lead for a set of optical lead parameters delivered to a target site in the brain of the patient.
